(19) United States
(12) Patent Application Publication
(10) Pub. No.: US 2007/0179585 A1
(43) Pub. Date: Aug. 2, 2007

(54) Method and apparatus for treating a wound

(76) Inventors: Mark Chandler, Southern Pines, NC (US); Andrea Moretti, London (GB)

Correspondence Address: DICKSTEIN SHAPIRO LLP, 1825 EYE STREET NW, Washington, DC, 20006-5403, US

(21) Appl. No.: 11/342,661

(22) Filed: Jan. 31, 2006

(51) Int. Cl. A61F 2/06

(52) U.S. Cl. 623/1.1

(57) ABSTRACT

A method of treating a diabetic foot or leg ulcer comprising the steps of placing a pair of electrodes placed spaced apart in the region of a diabetic foot or leg ulcer and applying a predetermined sequence of current waveforms across the electrodes. The sequence of current waveforms comprises a first waveform comprising a series of current pulses having an amplitude in a range of from 80 to 300 μA, having a frequency in a range from 0.5 to 1.5 pulses per second and a pulse width in a range from 333 to 1000 ms, a second waveform comprising a series of current pulses having an amplitude in a range of from 20 to 60 μA, having a frequency in a range from 2 to 4 pulses per second and a pulse width in a range from 125 to 250 ms and a third waveform comprising a series of current pulses having an amplitude in a range of from 250 to 640 μA, having a frequency in a range of from 80 to 120 pulses per second and a pulse width in a range from 4 to 6 ms. The electrodes may be placed in the region of the diabetic foot or leg ulcer in a manner that is compatible with the application of an off-loading boot or cast.

### FIELD OF THE INVENTION

The present invention generally relates to a method and apparatus for treating a wound involving application of electrical signals to the region of the wound. In particular, the invention relates to a method and apparatus for treating a diabetic foot or leg ulcer.

### BACKGROUND OF THE INVENTION

Chronic wounds such as diabetic foot or leg ulcers (DFU) which do not heal, represent a serious problem to sufferers and healthcare providers. The medical condition diabetes can cause damage to the nerve and vascular supply in the feet and legs. Such damage to the nerve supply or peripheral neuropathy leads to reduced or no sensation in the feet and lower legs. Consequently, sufferers may be unaware of injury caused to the feet by, for example ill-fitting footwear, an object in their shoes or walking on a piece of glass, stone or a drawing pin. Continued walking on the injured foot leads to further damage and minor lesions may develop into more serious wounds such as foot ulcers. Diabetic foot and leg ulcers occur at pressure points and are generally small but deep. Damage to the vascular supply or peripheral vascular disease results in the cut-off of the normal arterial supply by blockage of small vessels and delays healing of wounds or ulcers. Infection can then lead to the breakdown of tissue and spread to other parts of the foot or leg. Diabetic foot or leg ulcers are expensive to treat and in severe cases can lead to limb amputation. Early detection and appropriate treatment are very important in the management of the disease and can lead to the prevention of amputations.

Conventional treatment of diabetic foot or leg ulcers consists of dressing the wound with a suitable compound and then placing a dressing over the wound followed by the application of what is known as an “off-loading boot” or cast to reduce pressure applied to the wound. Sufferers are required to wear the off-loading boot when putting weight on the foot.

Studies have shown that the process of healing, growth and regeneration in living tissue is brought about by the flow of endogenous electrical current. It has been suggested that the application of external microcurrents to injured tissue can assist the body's natural healing process by augmenting the flow of current through the injured tissue. The application of electrical signals to injured tissue as a form of therapy is known as electrotherapy and has been described in various publications.

U.S. Pat. No. 4,982,742 describes a method and apparatus for facilitating the healing of soft tissue wounds involving the application of a single bi-phase microcurrent waveform to a selected area of tissue. The waveform is characterised by a frequency ranging from 10 to 50 Hz and an amplitude ranging between 100 and 1000 μA. The waveform is delivered by a disposable bandage containing an integrated circuit and power source.

Similarly the method described in U.S. Pat. No. 6,393,326 uses one waveform throughout treatment. The electrical treatment signal disclosed in this document is characterised by a bipolar voltage waveform at a frequency of between 2 Hz and 10 Hz. This method is particularly adapted to the treatment of bedsores which are known to have substantially zero electrical activity.

EP367320 also relates to a system for the treatment of wounds by electric stimulation. The document discloses a waveform generator adapted to generate either a direct current signal or a pulsed signal comprising pulses with a pulse width of less than 1 ms. It further discloses that optimal pulse width is about 0.1 ms. The DC current application is believed to produce wound healing and the pulse signals when applied directly into the wounds are said to produce a pain-relief effect.

None of the above methods is specifically adapted to the treatment of diabetic foot or leg ulcers.

There is therefore a recognised need for an effective method of electrotherapy tailored to the treatment of diabetic foot or leg ulcers and that is compatible with conventional treatment.

### SUMMARY OF THE INVENTION

Accordingly, the present invention provides a method of and an apparatus for treating a diabetic foot or leg ulcer.

In one aspect of the invention, a method of treating a diabetic foot or leg ulcer comprises placing a plurality of electrodes spaced apart in the region of the diabetic foot or leg ulcer and applying a sequence of predetermined waveforms between electrodes of the plurality of electrodes. The sequence of waveforms includes a first waveform comprising a series of current pulses having an amplitude of between 80 and 300 μA, having a frequency of between 0.5 and 1.5 pulses per second and a pulse width of between 333 and 1000 ms, a second waveform comprising a series of current pulses having an amplitude of between 20 and 60 μA, a frequency of between 2 and 4 pulses per second and a pulse width of between 125 and 250 ms, and a third waveform comprising a series of current pulses having an amplitude of between 250 and 640 μA, having a frequency of between 80 and 120 pulses per second and a pulse width of between 4 and 6 ms.

The application of waveforms with the ranges of parameters given above retards degeneration of diabetic foot or leg ulcers and increases efficacy in their healing. In particular, this aspect of the invention provides a new and improved electrotherapy treatment for diabetic foot or leg ulcers which addresses the two chief problems of peripheral neuropathy and peripheral vascular disease. Each phase of treatment has a particular purpose and the treatment enables the body's own natural healing mechanisms which have failed due to chronic disease or diminishment of blood flow to the area.

The first waveform works to decrease the resistance of the tissues, to reduce swelling (edema), inflammation, resistance of the wound, and to provide a migratory control for cells such as macrophages and fibroblasts. The second waveform works directly at the individual cellular level, increasing the cells production of protein, increasing cellular energy (ATP), and stimulating healthy cell division. This process addresses the problem of decreased sensation from peripheral neuropathy and provides the necessary proteins for strong wound healing and effective re-epithelialzation. The third waveform increases blood supply through a process known as angiogenesis—the creation of new small blood vessels. This process addresses the problem of decreased blood supply.

In an embodiment of the invention, the first waveform is applied over a period of time ranging from 5 to 10 minutes, the second waveform is applied over a period of time ranging from 10 to 20 minutes and the third waveform is applied over a period of time ranging from 20 to 40 minutes.

In another embodiment of the invention the second waveform is further applied over a period ranging from 1 hour to 3 hours. By further applying the second waveform for a period of 1 to 3 hours, the problem of decreased sensation, a significant problem in diabetic foot or leg ulcers, will be addressed during a longer period of time and the benefits of increased cell production of protein, increased cellular energy and stimulation of healthy cell division will be significantly increased. In addition since this phase of treatment mimics what the body does naturally, the tissue will be stimulated for longer and will thereby be enabled to trigger its own natural healing process.

A pause of between 3 and 15 hours wherein no waveform is applied may follow application of the third waveform. This time period between treatments is particularly advantageous in that it enables the body to respond to the treatment and to heal itself naturally.

In another embodiment of the invention, no waveform is applied for a period of between three hours and fifteen hours after the second waveform has been reapplied. A pause in which no waveform is applied is particularly advantageous after this phase of treatment since the body's own natural healing mechanism has been mimicked during a longer period of time during this phase and thus the body will be encouraged to heal itself naturally using its own natural mechanism after the phase of treatment has stopped.

In another embodiment of the invention, no waveform is applied for a period of between three hours and fifteen hours after the third waveform is applied and before the second waveform is reapplied.

In an embodiment of the invention the pause between treatments is approximately seven hours. This time period is beneficial in that it is sufficiently long enough to give the body time to heal itself and is not too long to allow toxins and wound resistance to build up in the average wound. In addition, by having a pause of an odd number of hours, individual treatments which are automatically timed from 3 days to 2 weeks will not always occur at the same time every day as the starting time of treatments will vary.

In another embodiment the sequence of waveforms is automatically repeated. This is advantageous in that the treatment may be delivered over long periods of time without constant intervention from trained medical personnel. This is highly beneficial to patients who struggle to find time to receive treatment.

In one embodiment, the polarity of the electrodes is reversed approximately every 5 to 15 seconds during application of the first waveform. In another embodiment, the polarity of the electrodes is reversed approximately every 5 to 15 seconds during application of the second waveform. In a further embodiment, the polarity of the electrodes is reversed approximately every 5 to 15 seconds during application of the third waveform. In an even further embodiment, the polarity of the electrodes is reversed approximately every 5 to 15 seconds during application of all the waveforms.

In one embodiment, the first waveform comprises a series of current pulses having an amplitude of substantially 100 μA, a frequency of substantially 1 pulse per second and a pulse width of substantially 500 ms, the second waveform comprises a series of current pulses having a amplitude of substantially 40 μA, a frequency of substantially 3 pulses per second and a pulse width of substantially 166 ms, the third waveform comprises a series of current pulses having an amplitude of substantially 320 μA, a frequency of substantially 100 pulses per second and a pulse width of substantially 5 ms.

In one embodiment, the first waveform is applied over a period of time of substantially 5 minutes, the second waveform is then applied over a period of time of 20 minutes, the third waveform is then applied over a period of time of substantially 30 minutes, no waveform is applied for a period of 7 hours and then the second waveform is applied over a period of time of substantially 2 hours.

In one embodiment, the pulses are substantially rectangular. This encompasses pulses which are functionally rectangular or square.

In an embodiment of the invention the electrodes are placed in the area of treatment in a manner compatible with the fitting of an off-loading arrangement over the area of treatment.

In an embodiment of the invention the electrodes are placed in a region peripheral to the diabetic foot or leg ulcer.

The advantage of applying electrical signals to the skin peripheral to the ulcer is that there is a significantly reduced risk of contaminating the wound and spreading infection, there is less discomfort and pain to the patient and it is more acceptable to and practical for nursing staff and other medical personnel to apply the electrodes to unbroken skin Moreover, applying electrical signals in the periwound area allows electrical current therapy to be administered through the regenerative tissue under the ulcer.

In an embodiment, each electrode of a pair of electrodes is positioned on opposite sides of the wound to one another so that the current passes under the wound and promotes the growth of regenerative tissue under the wound.

In an embodiment, each electrode is placed approximately 1 cm from an edge of the wound.

In a further embodiment, the end of each electrode extends beyond the outermost edges of the wound so that the entire surface of the wound is positioned between two electrodes.

In another embodiment, each end of each electrode extends beyond the outermost edges of the wound by approximately 1.0 to 1.5 cm.

In a further aspect of the invention an apparatus for treating a diabetic foot or leg ulcer according to the invention, includes a waveform generator adapted to generate: a first waveform comprising a series of current pulses having an amplitude in a range of from 80 to 300 μA, having a frequency in a range from 0.5 to 1.5 pulses per second and a pulse width in a range from 333 to 1000 ms, over a period of time in a range from 5 to 10 minutes; a second waveform comprising a series of current pulses having an amplitude in a range of from 20 to 60 μA, having a frequency in a range from 2 to 4 pulses per second and a pulse width in a range from 125 to 250 ms over a period of time in a range from 10 to 20 minutes; and a third waveform comprising a series of current pulses having an amplitude in a range of from 250 to 640 μA, having a frequency in a range of from 80 to 120 pulses per second and a pulse width in a range from 4 to 6 ms over a period of time in a range from 20 to 40 minutes; and the second waveform over a period of time in a range from 1 hour to 3 hours; and output connectors for connection to an electrode arrangement for applying the waveforms across the wound.

In one embodiment the waveform generator includes a switch arrangement for switching the polarity of the pulses.

In another embodiment the waveform generator is pre-programmed with one or more programs for generating one of said waveforms or a pre-determined sequence of said waveforms.

In another aspect of the invention a method of treating a diabetic foot or leg ulcer involves placing a plurality of electrodes spaced apart in the region of the ulcer in such a manner that an off-loading covering may be fitted on the foot and/or leg, placing an off-loading arrangement over the electrodes and the region of the ulcer to reduce the pressure in the area of the ulcer and applying an electrical current between electrodes of the plurality of electrodes. This aspect of the invention provides simultaneous conventional off-loading treatment and electrotherapy treatment to provide an improved method of treating diabetic foot or leg ulcers.

In an embodiment of this aspect of the invention the electrodes are placed in a region peripheral to the diabetic foot or leg ulcer.

Electrical stimulation programs for wound healing in the past have all utilized an electrode placed inside the wound bed with a peripheral large return electrode positioned elsewhere on the leg. In such systems, the electrode used inside the wound bed is by necessity bulky and irregular, and made to fit the often deep cavity of the wound. The top of the electrode where contact is made with a connecting wire is often located several centimetres above the wound. The bulky and irregular shape and size resulting from such an arrangement makes it impossible to apply and secure a close fitting boot or cast. Attempting to fit a close-fitting boot or cast with this configuration of electrodes, would transfer the pressure point to the top of the wound itself, effectively making the off-loading boot or cast totally ineffective if not harmful. Moreover, the electrodes cannot be left in the wound and must be removed immediately after treatment in order to avoid contamination of the wound. This necessitates the removal of the boot or cast for each treatment (at least once per day) which is impractical and inconvenient.

By applying electrodes to the periphery of the wound over intact skin utilizing thin electrodes, the above problems are eliminated. A tight fitting boot or cast will not affected by the electrodes which can thus be left in place for days at a time. Consequently, many treatments lasting a week or for a longer period of time can be accomplished without removing the boot or cast.

Such an arrangement has benefits for both the patient and care provider. As mentioned above, with previously described electrical stimulation devices, the boot or cast would have to be removed every time a stimulation treatment was done, sometimes as often as twice a day. This is cumbersome and inconvenient to the patient and often leads to frequent non-compliance in wearing the boot. Moreover, an off loading cast which is described in many studies as being more efficacious than an off loading boot is not an option for the physician to use. With the current invention, casting can be easily applied in conjunction with electrical stimulation and off-loading boots, which are difficult to remove and reapply properly, can be left in place for days, maximizing their benefit.

The method of treating a wound according to the invention has the advantage that it consists of different treatment phases adapted to specific problems associated with diabetic foot or leg ulcers. In addition, it is advantageous to have a method for promoting the healing of wounds that is non-invasive and painless, that is easy to apply and that is capable of being used on a long term basis without continual intervention from skilled medical personnel.

### BRIEF DESCRIPTION OF THE DRAWINGS

Embodiments of the invention will now be described with reference to the accompanying drawings, in which:

FIG. 1 is a perspective view of a device for generating electrical waveforms according to an embodiment of the present invention;

FIG. 2 is a schematic diagram showing one channel of a device for generating electrical signals according to the embodiment of the present invention;

FIG. 3 is a schematic diagram of a waveform generator for generating waveforms according to the embodiment of the present invention;

FIG. 4 is a schematic diagram of the area of treatment showing the disposition of electrode pads according to the embodiment of the present invention;

FIG. 5a is a graphical illustration of a first waveform generated by the embodiment of the present invention;

FIG. 5b is a graphical illustration of a second waveform generated by the embodiment of the present invention;

FIG. 5c is a graphical illustration of a third waveform generated by the embodiment of the present invention; and

FIG. 6 is a simplified illustration of a method of treating a diabetic foot or leg ulcer according to a further embodiment of the invention

### DESCRIPTION OF THE EMBODIMENTS

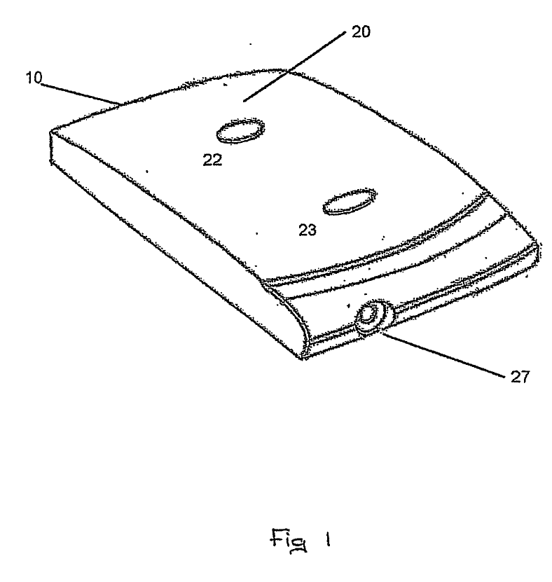

FIG. 1 is a perspective view of a device 10 for applying electrical signals to an area of tissue according to an embodiment of the present invention. The electrotherapy device 10 comprises a housing 20, an electrode port 27, an input switch 23 and an on/off switch 22. The input switch 23 and the on/off switch 22 may be the push button type. The housing 20 encloses a channel 30.

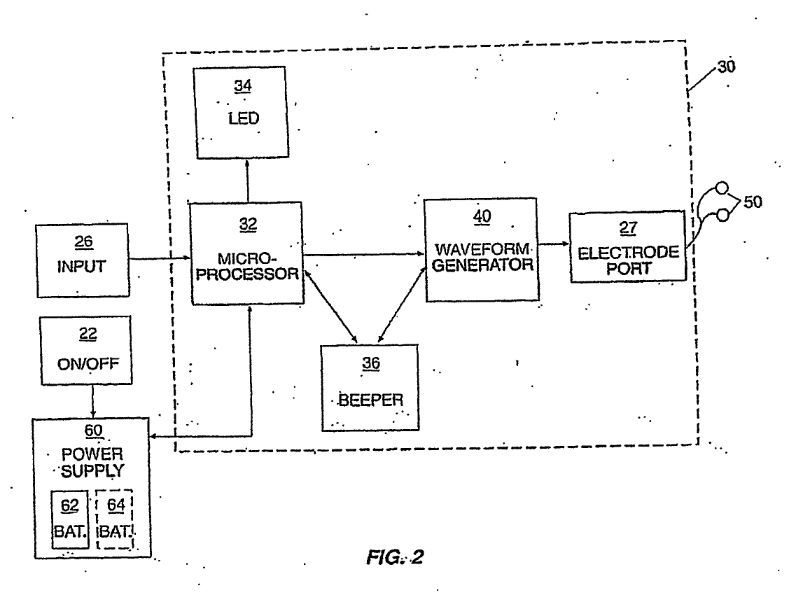

FIG. 2 is a schematic diagram of the device 10 showing the channel 30. The channel 30 includes an electrode port 27, a microprocessor 32, a waveform generator 40, LEDs 34 and a beeper 36. The channel 30 is connected to the on/off switch 22, input switch 23, a power supply 60 and a pair of electrodes 50. The electrodes 50 may be of any type known in the art of electrotherapy. The power supply 60 supplies the microprocessor and the rest of the channel 30 with power. The power supply includes a battery which supplies power to the channel 30. Turning on the device 10 via the on/off switch 22 activates the power supply 60, which in turn controls the on/off stage of the battery 62. In this embodiment the power supply converts the battery voltage to a supply logic level of five volts.

The microprocessor 32 controls and/or monitors voltage, input switch 23, status LEDs 34, beeper 36 and the waveform generator 40. The waveform generator 40 receives signals from the microprocessor 32, transforms them into the appropriate current waveforms, and supplies the waveforms to the electrode port 27. The electrodes 50 transfer the waveforms from the electrode port 27 to the tissue to be treated. Input switch 23 and on/off switch 22 are resistor multiplexed into an analog port of the microprocessor 32.

The status of the device 10 is indicated by LEDs 34, controlled by microprocessor 32. Beeper 36 is activated when the device 10 detects high resistance between the individual electrodes of the electrode pair 50, indicating that the electrodes 50 are not making proper contact with the portion of the body to be treated. Such a situation is called a pad open condition. Beeper 36 is also activated when a low battery voltage is detected.

The device 10 is activated via on/off switch 22. Once energized, the microprocessor checks switch 23. Switch 23 is used to start a pre-programmed three stage waveform treatment program. The microprocessor 32 sends appropriate signals to the waveform generator 40 based on the pre-programmed three stage waveform treatment program to cause the appropriate signals to be sent to the electrodes 50. The microprocessor also instructs the LEDs and the beeper 36 to indicate the appropriate status. The device automatically cycles through the three stage treatment program when switch 23 is pressed, and automatically switches off when the cycle of treatment stages has finished. During the treatment program a ticking noise is emitted by the device 10 to indicate that the program is running. The beeper 36 emits a series of beeps at the end of the treatment program to indicate that the program has finished.

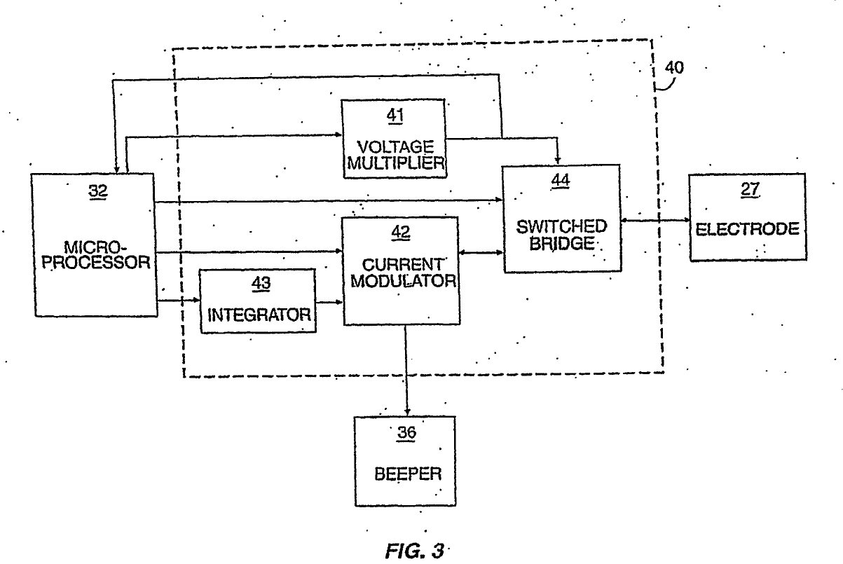

The waveform generator 40 is shown in more detail in FIG. 3. The waveform generator comprises a voltage multiplier 41, a current modulator 42, an integrator 43, and a switched bridge 44. The combined elements of the waveform generator 40 take power from the power supply 60 and generate a current waveform under control of the microprocessor 32.

The voltage multiplier supplies a voltage pumped signal to the switched bridge 44. In this embodiment, the voltage multiplier multiplies the battery voltage by 6. The voltage multiplier includes a voltage feedback loop with the microprocessor 32. The feedback voltage is fed to an ADC and software reduces the drive frequency to reduce the output voltage as required.

The switched bridge 44 supplies the generated current waveform to the electrode port 27. In this embodiment, the switched bridge 44 comprises four opto-isolators in a bridge configuration. In addition to the voltage pumped signal from the voltage multiplier 41, the switched bridge receives a polarity control signal from the microprocessor 32 and a current modulation signal from the current modulator 42. The integrator 42 processes the waveform signals received from the microprocessor 32 resulting in ramp, sine and square wave outputs as required. These outputs are sent to the current modulator 42. The current modulator 42 controls the output current level under direction of the microprocessor 32. The current range is controlled by a software-switchable sense resistor. The current modulator 42 receives signals from the integrator 43 and also receives current control signals from the microprocessor 32.

The microprocessor 32 supplies various signals to various portions of the waveform generator 40 so as to generate appropriate current waveforms. For example, the microprocessor 32 supplies a modulated square wave signal to the voltage multiplier 41, an output polarity setting to the switched bridge 44, a pulse width modulated synthesized waveform to the integrator 43 and a current level selection signal to the current modulator 42. The waveform parameters are stored in an EPROM and cannot be modified by the user. The microprocessor can be considered as being functionally part of the waveform generator. The waveform generator 40 supplies electrical signals to electrodes 50 via the electrode port 27.

In alternative embodiments the device may include two or more channels for simultaneously transmitting electrical signals to two or more electrode ports. A second channel may communicate with the first channel through an opto-isolator.

In further embodiments of the invention, the device 10 may include a display. In further embodiments, the microprocessor and the waveform generator may constitute one unit.

In yet further embodiments the device 10 may be programmed with two or more waveform treatment programs for generating a predetermined waveform or a predetermined sequence of waveforms. The device may further include further input switches to select between different waveform treatment programs.

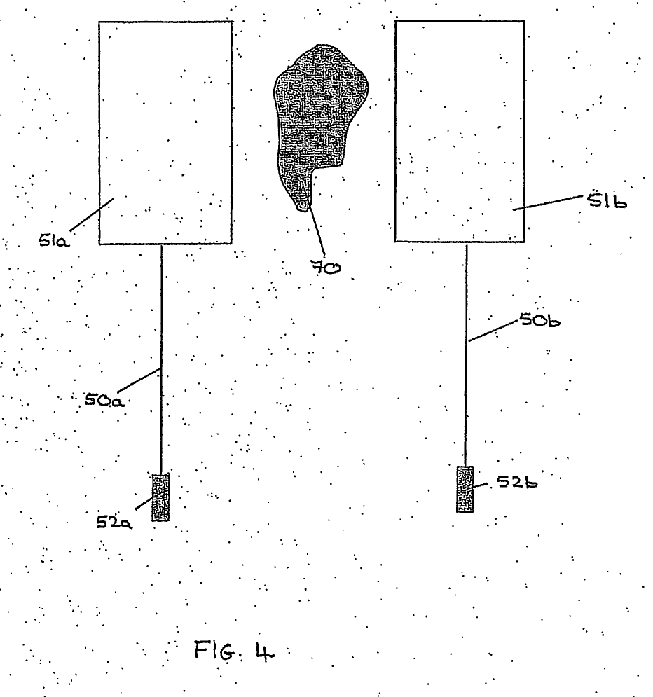

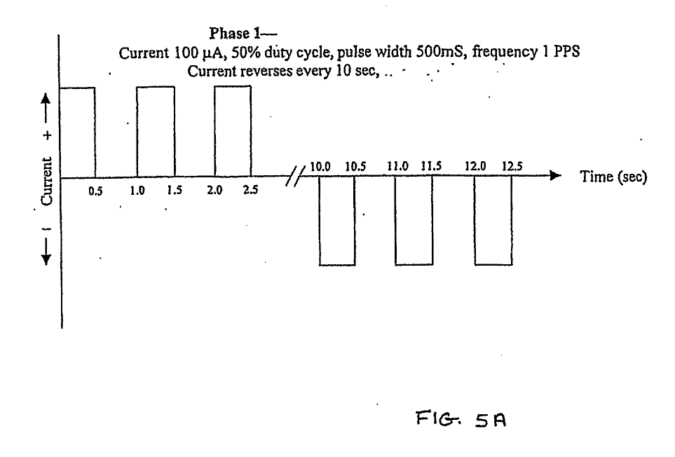

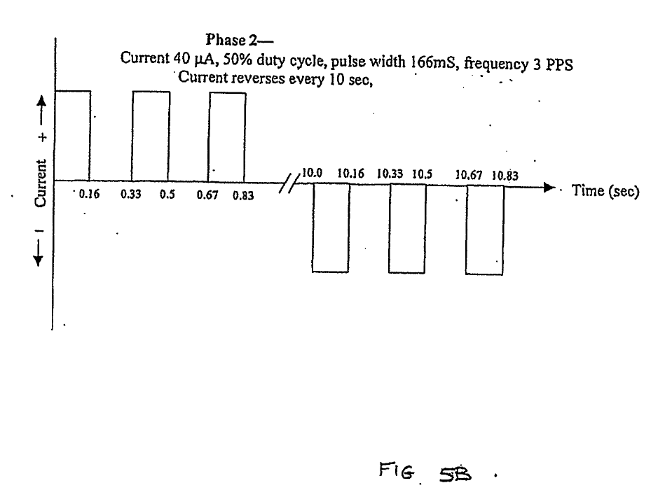

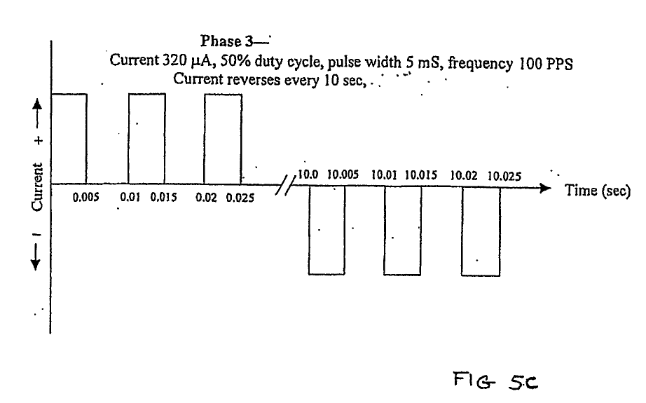

A method of treatment in accordance with an embodiment of the present invention will now be described with reference to FIGS. 4 to 5.

The method of the present invention includes steps of arranging electrodes around the ulcer to be treated, providing a first electrotherapy waveform during a first treatment stage, providing a second electrotherapy waveform during a second treatment stage and providing a third electrotherapy waveform during a third treatment stage. The second treatment stage is then repeated.

The electrical waveforms are administered to an area of a body via a pair of electrode pads 51a and 51b which are placed on the surface of the unbroken skin on opposite sides of an ulcer 70 substantially parallel to the longitudinal axis of the ulcer as shown in FIG. 4. The electrode pads adhere to the skin of the patient and disperse current evenly across the surface of the skin with which they are in contact. The electrode pads may be of any type known in electrotherapy and may be available in different sizes. The inner edge of the electrode pads are placed approximately 1 cm from the outer edges of the ulcer. The electrode pads extend approximately 1.5 cm from the outer edges of the ulcer in both directions substantially parallel to the longitudinal axis of the ulcer. The electrode pads are connected to a pair of electrode leads 50a and 50b which each have a connector 52a and 52b, respectively, at one end for connection to the device 10. Since the electrodes are placed outside the ulcer there is no need to remove and reapply any dressing on the ulcer and the ulcer is not irritated by contact with the electrode pads.

In a first stage of treatment the waveform illustrated in FIG. 5a is applied to the treatment area. The first treatment stage is particularly suited to reducing the resistance of the injured tissue. It has been proposed that injured tissue has a higher electrical resistance than healthy tissue such that the flow of natural electrical current through an injured section of the body is lower than the flow through normal surrounding tissue. The decreased electrical flow through the injured tissue decreases the cellular capacitance. Consequently, healing of the injured tissue is impaired. It has been further proposed that reducing the resistance of injured tissue and allowing the body's natural bio-electricity to enter the area would aid the healing process or reduce pain. To facilitate a change in tissue resistance the electrodes are provided with a waveform comprising a series of current pulses with an amplitude of 100 μA, having a frequency of 1 pulse per second (pps) and a pulse width of 500 ms. The pulses are substantially square and are characterised by a rapid rise to a current level, a hold at that current level, followed by a rapid return to near zero current. The polarity of the electrodes is reversed at periodic intervals of approximately 10 seconds. This stage of treatment lasts for 5 minutes. This stage of treatment helps to decrease the resistance of the tissues, to reduce swelling (edema) and inflammation, and to provide migratory control for cells such as macrophages and fibroblasts.

In a second stage of treatment the waveform illustrated in FIG. 5b is applied to the treatment area. The second stage of treatment is particularly suited to healing injured tissue by providing a current that mimics the body's natural current. To facilitate healing of the injured tissue the electrodes are provided with a waveform comprising a series of pulses with an amplitude of 40 μA, having a frequency of 3 pps and a pulse width of 166 ms. The polarity of the electrodes is reversed at periodic intervals of approximately 10 seconds. The second stage of treatment lasts for 20 minutes when applied as part of a three phase treatment and lasts for 2 hrs when applied on its own. The current applied to the tissue at this stage of treatment works directly ay an individual cellular level, —increasing the cells production of protein, increasing cellular energy (ATP), and stimulating healthy cell division. Decreased sensation in the lower legs and feet from peripheral neuropathy is addressed by this stage of treatment.

In a third stage of treatment the waveform illustrated in FIG. 5c is applied to the treatment area. The third stage of treatment is particularly suited to promoting blood vessel regeneration (angiogenesis). To facilitate blood vessel regeneration in injured tissue the electrodes are provided with a waveform comprising a series of pulses with an amplitude of 320 μA, having a frequency of 100 pps and a pulse width of 5 ms. The third treatment stage lasts for 30 minutes.

The three treatment stages are firstly automatically executed sequentially. The second treatment stage follows the first treatment stage and the third treatment stage follows the second treatment stage. After sequential execution of the three treatment stages no waveforms are applied for a period of seven hours. This break in treatment allows the body to begin healing itself naturally. After the seven hour break in treatment, stage 2 of the treatment is reapplied for a period of 2 hours. This stage of treatment is followed by another seven hour duration where no waveforms are applied to the area of treatment. A pause is particularly advantageous after this phase of treatment since the body's own natural healing mechanism has been mimicked during a longer period of time during this phase and thus the body will be encouraged to heal itself naturally using its own natural mechanism after the phase of treatment has stopped.

The cycle of treatment phases and pauses in treatment can be repeated automatically for a period of 3 days to a period of 2 weeks.

Since the cycle of treatment stages and pauses in treatment is executed automatically there is no need for further user interaction beyond starting the treatment program cycle. The patient is free to relax and read a book or watch television while receiving treatment. The treatment can be administered by the patient himself in the comfort of his own home without the need to go to hospital. An advancement whereby the device is attached to the leg and requires zero interference by the patient as the device delivers currents automatically twice a day.

In a further embodiment of the invention, a plurality of pairs of electrodes may be placed around the ulcer. The inner edge of the electrode pads may be placed at different distances from the outer edges of the ulcer. Although in the embodiment described above the electrode pads extend beyond the area of the ulcer, in alternative embodiments the electrodes may not extend beyond the area of the ulcer. In an alternative embodiment of the invention three electrodes may be placed around the ulcer and the current waveforms may be applied between different electrodes of the three electrodes. In further embodiments of the invention four or more electrodes may be placed around the ulcer and the current waveforms may be applied between different electrodes simultaneously or sequentially.

In alternative embodiments of the invention, the waveform applied during the first treatment stage comprises a series of current pulses having an amplitude in a range of from 80 to 300 μA, having a frequency in a range from 0.5 to 1.5 pulses per second and a pulse width in a range from 333 to 1000 ms, the waveform applied during the second stage of treatment comprises a series of current pulses having an amplitude in a range of from 20 to 60 μA, having a frequency in a range from 2 to 4 pulses per second and a pulse width in a range from 125 to 250 ms, and the waveform applied during the third stage of treatment comprises a series of current pulses having an amplitude in a range of from 250 to 640 μA, having a frequency in a range of from 80 to 120 pulses per second and a pulse width in a range from 4 to 6 ms.

In further embodiments of the invention, the first waveform is applied over a period of time ranging from 5 to 10 minutes, the second waveform is applied over a period of time ranging from 10 to 30 minutes, the third waveform is applied over a period of time ranging from 20 to 40 minutes and the second waveform is further applied over a period ranging from 1 hour to 3 hours. The treatment can be thus tailored to the type, size and condition of the wound to be treated.

In alternative embodiments of the invention the polarity of the electrodes may be reversed at periodic intervals of approximately 5 to 15 seconds. In further embodiments the polarity of the electrodes may not be reversed.

Although in this embodiment of the invention the pause between treatments is 7 hours the range of time between treatments can be altered depending on the type, size and condition of the wound to be treated.

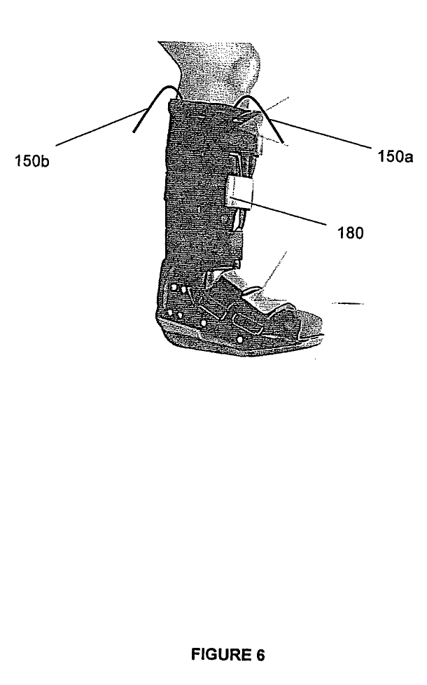

The method of the present invention according to this embodiment is particularly suited to the treatment of diabetic foot or leg ulcers. In a further embodiment of the invention, electrodes pads are placed around the wound on the foot or the leg as described above and the method further includes the step of fitting the foot or leg with a close fitting off-loading boot 180 as shown in FIG. 6. Electrode pads with a thickness of a few millimetres are used for compatibility with the fitting of the off loading boot. This allows the off-loading boot to be applied without creating a potentially harmful pressure point in the area of treatment. In particular, since the electrodes are positioned in the periphery of the ulcer there is no risk of creating a potentially harmful pressure point in the ulcer itself. Electrode leads 150a and 150b connected to the electrode pads protrude from the off-loading boot 180 for connection to an electrotherapy device (not shown). The electrode leads (150a and 150b) are between 5 cm and 50 cm in length. Waveforms as described hereinbefore are applied between the electrodes. In this embodiment of the invention, a conventional off-loading boot is used. The off loading boot off-loads the pressure from the area of the ulcer helping it to heal. Since the electrode connectors protrude from the bandaging they are easily accessible for connection to the electrotherapy device and there is no need to remove and refit the boot before and after the electrotherapy treatment. Moreover, since none of the electrodes are in contact with the ulcer there is no need to regularly remove the electrodes to avoid contamination of the ulcer. Since there is no need to remove the off-loading boot each time treatment is administered this is practical for both the patient receiving treatment and the carder administering the treatment. Furthermore, the electrotherapy treatment works in combination with the off loading treatment providing benefits of the two treatments simultaneously to the patient.

Although there is no need to remove the off-loading boot each time electrotherapy treatment is applied, the patient is able to remove the boot easily at night, while the pads remain attached to the foot or leg.

Alternatively, the patient may be fitted with a molded cast for off loading the pressure from the ulcer. In this case the electrode dressing is placed around the ulcer before the cast is applied. The cast is then applied as normal over the electrode system, with the electrical generator strapped to the outside of the cast and with connection to the electrode cables made after threading the electrode cable under or through the cast.

In further embodiments other off-loading arrangements known in the art for dispersing the pressure in the area of the ulcer may be used. Such arrangements may comprise one or more components performing the function of dispersing pressure from the wound. Off loading devices or coverings known in the art include, for example, off-loading total contact casts, removable off-loading boots, off-loading wedges, shoes, sandals, walking braces, casting socks, casting slippers, prefabricated walkers, ortho tics,healing shoes and sandals, post-operative shoes, diabetic shoes, custom-molded insole shoes, shoe inserts, etc. Specific products include but are not limited to the CROW (Chariot Restraint Ortho tic Walker) Boot, the OH pressure relief Walker, CAM Walkers, Aircasts, D.H. Walkers, Royce Walkers, I.P.O.S. Shoes, reverse IPOS, PIB braces Darco Wedges and Ortho wedges., the Conformer Boot, Hope walking sandal, the Optima slipper, MABAL shoe, and Scotch-cast bootees.

Although FIG. 6 shows the electrode leads protruding from the top of the off-loading boot, in alternative embodiments the electrode leads may protrude from the side of the boot in the region of the ulcer so that the electrode leads do not have to be pressed along the length of the leg.

It will be appreciated that the electrical generator used to apply current to the electrodes positioned under the boot or cast can be any electrical stimulation device known in the field including but not limited to low volt pulsed galvanic, high volt pulsed galvanic, low intensity direct current, and pulsed milli-ampere current generators.

It will also be appreciated that while optimal electrical waveforms have been described above, the concept of using electrical stimulation as an adjunct to off-loading therapy can be used with all electrical waveforms.

Although the present invention has been described with reference to specific embodiments, it will be apparent to a skilled person in the art that modifications lie within the spirit and scope of the present invention.

1. A method of treating a diabetic foot or leg ulcer, comprising:
placing a plurality of electrodes spaced apart in the region of the diabetic foot or leg ulcer; and
applying a sequence of current waveforms between electrodes of the plurality of electrodes to treat the diabetic foot or leg ulcer, the sequence of current waveforms comprising:
a first waveform comprising a series of current pulses having an amplitude in a range of from 80 to 300 μA, having a frequency in a range from 0.5 to 1.5 pulses per second and a pulse width in a range from 333 to 1000 ms;
a second waveform comprising a series of current pulses having an amplitude in a range of from 20 to 60 μA, having a frequency in a range from 2 to 4 pulses per second and a pulse width in a range from 125 to 250 ms; and
a third waveform comprising a series of current pulses having an amplitude in a range of from 250 to 640 μA, having a frequency in a range of from 80 to 120 pulses per second and a pulse width in a range from 4 to 6 ms.

2. A method of treating a diabetic foot or leg ulcer according to claim 1, wherein the first waveform is applied over a period of time ranging from 5 to 10 minutes.

3. A method of treating a diabetic foot or leg ulcer according to claim 1, wherein the second waveform is applied over a period of time ranging from 10 to 30 minutes.

4. A method of treating a diabetic foot or leg ulcer according to claim 1, wherein the third waveform is applied over a period of time ranging from 20 to 40 minutes.

5. A method of treating a diabetic foot or leg ulcer according to claim 1, wherein the second waveform is applied over a period ranging from 1 hour to 3 hours.

6. A method of treating a diabetic foot or leg ulcer according to claim 1, wherein the first waveform is applied over a period of time ranging from 5 to 10 minutes, the second waveform is applied over a period of time ranging from 10 to 30 minutes and the third waveform is applied over a period of time ranging from 20 to 40 minutes.

7. A method of treating a diabetic foot or leg ulcer according to claim 6, wherein the second waveform is further applied over a period of time ranging from 1 hour to 3 hours.

8. A method of treating a diabetic foot or leg ulcer according to claim 6, wherein no waveform is applied for a period of time ranging from 3 hours to 15 hours after the third waveform is applied.

9. A method of treating a diabetic foot or leg ulcer according to claim 7, wherein no waveform is applied for a period of time ranging from 3 hours to 15 hours after the second waveform is further applied.

10. A method of treating a diabetic foot or leg ulcer according to claim 9 wherein no waveform is applied for a period of time ranging from 3 hours to 15 hours after the third waveform is applied and before the second waveform is reapplied.

11. A method of treating a diabetic foot or leg ulcer according to claim 8, wherein no waveform is applied for a period of time of substantially 7 hours after the third waveform is applied.

12. A method of treating a diabetic foot or leg ulcer according to claim 9, wherein no waveform is applied for a period of time of substantially 7 hours after the second waveform is further applied.

13. A method of treating a diabetic foot or leg ulcer according to claim 10, wherein the sequence of waveforms is automatically repeated.

14. A method of treating a diabetic foot or leg ulcer according to claim 6, wherein the first waveform is applied over a period of time of substantially 5 minutes, the second waveform is applied over a period of time of substantially 20 minutes and the third waveform is applied over a period of time of substantially 30 minutes.

15. A method of treating a diabetic foot or leg ulcer according to claim 7 wherein the second waveform is further applied over a period of time of substantially 2 hours.

16. A method of treating a diabetic foot or leg ulcer according to claim 1, wherein the first waveform comprises a first part comprising said pulses of a first polarity and a second part comprising pulses of a second polarity.

17. A method of treating a diabetic foot or leg ulcer according to claim 1, wherein the second waveform comprises a first part comprising said pulses of a first polarity and a second part comprising pulses of a second polarity.

18. A method of treating a diabetic foot or leg ulcer according to claim 1, wherein the third waveform comprises a first part comprising said pulses of a first polarity and a second part comprising pulses of a second polarity.

19. A method of treating a diabetic foot or leg ulcer according to claim 1, wherein the first waveform comprises a first part comprising said pulses of a first polarity and a second part comprising pulses of a second polarity, the second waveform comprises a first part comprising said pulses of a first polarity and a second part comprising pulses of a second polarity and the third waveform comprises a first part comprising said pulses of a first polarity and a second part comprising pulses of a second polarity.

20. A method of treating a diabetic foot or leg ulcer according to claim 1, wherein during application of the first waveform the polarity of the electrodes is reversed approximately every 5 to 15 seconds.

21. A method of treating a diabetic foot or leg ulcer according to claim 1, wherein during application of the second waveform the polarity of the electrodes is reversed approximately every 5 to 15 seconds.

22. A method of treating a diabetic foot or leg ulcer according to claim 1, wherein during application of the third waveform the polarity of the electrodes is reversed approximately every 5 to 15 seconds.

23. A method of treating a diabetic foot or leg ulcer according to claim 1, wherein during application of the first waveform the polarity of the electrodes is reversed approximately every 5 to 15 seconds, during application of the second waveform the polarity of the electrodes is reversed approximately every 5 to 15 seconds and during application of the third waveform the polarity of the electrodes is reversed approximately every 5 to 15 seconds.

24. A method of treating a diabetic foot or leg ulcer according to claim 20, wherein during application of the first waveform the polarity of the electrodes is reversed at substantially every 10 seconds.

25. A method of treating a diabetic foot or leg ulcer according to claim 21, wherein during application of the second waveform the polarity of the electrodes is reversed at substantially every 10 seconds.

26. A method of treating a diabetic foot or leg ulcer according to claim 22, wherein during application of the third waveform the polarity of the electrodes is reversed at substantially every 10 seconds.

27. A method of treating a diabetic foot or leg ulcer according to claim 23, wherein during application of the first waveform the polarity of the electrodes is reversed at substantially every 10 seconds, during application of the second waveform the polarity of the electrodes is reversed at substantially every 10 seconds and during application of the third waveform the polarity of the electrodes is reversed at substantially every 10 seconds.

28. A method of treating a diabetic foot or leg ulcer according to claim 1, wherein:
the first waveform comprises a series of current pulses having an amplitude of substantially 100 μA, a frequency of substantially 1 pulse per second and a pulse width of substantially 500 ms;
the second waveform comprises a series of current pulses having a amplitude of substantially 40 μA, a frequency of substantially 3 pulses per second and a pulse width of substantially 166 ms;
the third waveform comprises a series of current pulses having an amplitude of substantially 320 μA, a frequency of substantially 100 pulses per second and a pulse width of substantially 5 ms.

29. A method of treating a diabetic foot or leg ulcer according to claim 1, wherein the plurality of electrodes are placed in contact with skin in a region peripheral to the diabetic foot or leg ulcer.

30. A method of treating a diabetic foot or leg ulcer according to claim 29, wherein each electrode of a pair of electrodes is positioned on opposite sides of the diabetic foot or leg ulcer to one another.

31. A method of treating a diabetic foot or leg ulcer according to claim 29, wherein each electrode is placed approximately 1 cm from an edge of the diabetic foot or leg ulcer.

32. A method of treating a diabetic foot or leg ulcer according to claim 29, wherein each end of each electrode extends beyond the outermost edges of the diabetic foot or leg ulcer.

33. A method of treating a diabetic foot or leg ulcer according to claim 32, wherein each end of each electrode extends beyond the outermost edges of the diabetic foot or leg ulcer by approximately 1 to 1.5 cm.

34. A method of treating a diabetic foot or leg ulcer according to claim 1, wherein the pulses are substantially rectangular.

35. A method of treating a diabetic foot or leg ulcer according to claim 1, further comprising placing an off-loading arrangement over the electrodes and the region of the foot ulcer to reduce the pressure in the area of the ulcer.

36. An apparatus for treating a diabetic foot or leg ulcer, the apparatus comprising:
a waveform generator adapted to generate:
a first waveform comprising a series of current pulses having an amplitude in a range of from 80 to 300 μA, having a frequency in a range from 0.5 to 1.5 pulses per second and a pulse width in a range from 333 to 1000 ms, over a period of time in a range from 5 to 10 minutes;
a second waveform comprising a series of current pulses having an amplitude in a range of from 20 to 60 μA, having a frequency in a range from 2 to 4 pulses per second and a pulse width in a range from 125 to 250 ms over a period of time in a range from 10 to 30 minutes; and
a third waveform comprising a series of current pulses having an amplitude in a range of from 250 to 640 μA, having a frequency in a range of from 80 to 120 pulses per second and a pulse width in a range from 4 to 6 ms over a period of time in a range from 20 to 40 minutes;
and the second waveform over a period of time in a range from 1 hour to 3 hours; and
output connectors for connection to an electrode arrangement for applying the waveforms across the ulcer.

37. An apparatus for treating a diabetic foot or leg ulcer according to claim 36, wherein said waveform generator includes a switch arrangement for switching the polarity of the pulses.

38. An apparatus for treating a diabetic foot or leg ulcer according to claim 36, wherein the waveform generator is pre-programmed with one or more programs for generating one of said waveforms or a pre-determined sequence of said waveforms.

39. An apparatus for treating a diabetic foot or leg ulcer according to claim 38, further comprising a user interface for selecting one of said waveforms or a predetermined sequence of said waveforms.

40. An apparatus for treating a diabetic foot or leg ulcer, comprising:
a pair of electrodes; and
means to generate a waveform for applying across said pair of electrodes; wherein the means to generate a waveform is adapted to generate a sequence of waveforms comprising:
a first waveform comprising a series of current pulses having an amplitude in a range of from 80 to 300 μA, having a frequency in a range from 0.5 to 1.5 pulses per second and a pulse width in a range from 333 to 1000 ms over a period of time in a range from 5 to 10 minutes;
a second waveform comprising a series of current pulses having an amplitude in a range of from 20 to 60 μA, having a frequency in a range from 2 to 4 pulses per second and a pulse width in a range from 125 to 250 ms over a period of time in a range from 10 to 30 minutes; and
a third waveform comprising a series of current pulses having an amplitude in a range of from 250 to 640 μA, having a frequency in a range of from 80 to 120 pulses per second and a pulse width in a range from 4 to 6 ms over a period of time in a range from 20 to 40 minutes;
and the second waveform over a period of time in a range from 1 hour to 3 hours.

41. An apparatus for treating a diabetic foot or leg ulcer according to claim 40, further comprising polarity switching means.

42. An apparatus for treating a diabetic foot or leg ulcer according to claim 41 further comprising user interface means for selecting one of said waveforms or a predetermined sequence of said waveforms.

43. A method of treating a diabetic foot or leg ulcer, comprising:
positioning a plurality of electrodes spaced apart in the region of a diabetic foot or leg ulcer in such a manner that an off-loading arrangement may be fitted on the leg and/or foot;
placing an off-loading arrangement over the electrodes and the region of the said foot ulcer to reduce the pressure in the area of the ulcer; and
applying an electrical current between electrodes of the plurality of electrodes.

44. A method of treating a diabetic foot or leg ulcer, according to claim 43 wherein the plurality of electrodes are placed in contact with skin in a region peripheral to the diabetic foot or leg ulcer.

45. A method of treating a diabetic foot or leg ulcer, according to claim 43 wherein applying an electrical current between electrodes of the plurality of electrodes comprises the steps of applying a first waveform comprising a series of current pulses having an amplitude in a range of from 80 to 300 μA, having a frequency in a range from 0.5 to 1.5 pulses per second and a pulse width in a range from 333 to 1000 ms for a period of time ranging from 5 to 10 minutes; a second waveform comprising a series of current pulses having an amplitude in a range of from 20 to 60 μA, having a frequency in a range from 2 to 4 pulses per second and a pulse width in a range from 125 to 250 ms for a period of time ranging from 10 to 30 minutes; and a third waveform comprising a series of current pulses having an amplitude in a range of from 250 to 640 μA, having a frequency in a range of from 80 to 120 pulses per second and a pulse width in a range from 4 to 6 ms for a period of 20 to 40 minutes.

46. A method of treating a diabetic foot or leg ulcer according to claim 45, wherein the second waveform is further applied over a period ranging from 1 hour to 3 hours.

47. A method of treating a diabetic foot or leg ulcer according to claim 45, wherein no waveform is applied for a period of time ranging from 3 hours to 15 hours after the third waveform is applied.

48. A method of treating a diabetic foot or leg ulcer according to claim 46, wherein no waveform is applied for a period of time ranging from 3 hours to 15 hours after the second waveform is further applied.

49. A method of treating a diabetic foot or leg ulcer according to claim 48 wherein no waveform is applied for a period of time ranging from 3 hours to 15 hours after the third waveform is applied and before the second waveform is reapplied.

50. A method of treating a diabetic foot or leg ulcer according to claim 47, wherein no waveform is applied for a period of time of substantially 7 hours after the third waveform is applied.

51. A method of treating a diabetic foot or leg ulcer according to claim 48, wherein no waveform is applied for a period of time of substantially 7 hours after the second waveform is further applied.

52. A method of treating a diabetic foot or leg ulcer according to claim 49, wherein the sequence of waveforms is automatically repeated.
